(19) United States
(12) Patent Application Publication
(10) Pub. No.: US 2025/0019683 A1
(43) Pub. Date: Jan. 16, 2025

(54) TRITIRACHIUM ALBUM PROTEINASE K MUTANT AND ITS ZYMOGEN, EXPRESSION PLASMID, RECOMBINANT PICHIA PASTORIS STRAIN AND METHOD OF PRODUCING THE MATURE FORM OF PROTEINASE K MUTANT

(71) Applicant: BLIRT S.A., Gdansk (PL)

(72) Inventors: Robert BRODZIK, Gdansk (PL); Arkadiusz POPINIGIS, Gdansk (PL); Justyna LEIBNER-CISZAK, Borkowo (PL); Dominik ZIETKOWSKI, Gdansk (PL); Krzysztof KUR, Reda (PL)

(21) Appl. No.: 18/711,923

(22) PCT Filed: Nov. 25, 2022
(86) PCT No.: PCT/PL2022/050084

§ 371 Date: May 21, 2024

(30) Foreign Application Priority Data
Nov. 26, 2021 (PL) P.439656

(51) Int. Cl. C12N 9/58

(52) U.S. Cl. CPC C12N 9/58; C12Y 304/21064

(57) ABSTRACT

A mutant of the Tritirachium album proteinase K zymogen containing a mutation that increases the efficiency of expression in yeast cells and facilitates the inactivation of mature proteinase K under stress and a method of obtaining it.

The present invention related to a Tritirachium album proteinase K mutant and its zymogen, an expression plasmid, a recombinant Pichia pastoris strain and a method of producing the mature form of proteinase K mutant.

Proteinase K (E.C. 3.4.21.64), a serine protease, is a proteolytic enzyme synthesised by the mould cells of Tritirachium album (Engyodontium album) ATCC 22625. Its analogues can also be found in bacterial cells, e.g. Serratia sp., Pseudoalteromonas sp., Alteromonas sp., Thermus sp., Vibrio sp, etc.

Depending on the source from which they are derived, these enzymes differ in molecular weight, length of the C-terminal domain, optimal temperature and pH values, as well as the demand for metal ions necessary for enzyme activity and/or stabilisation of its structure. The activity of enzymes from the proteinase K family usually increases when denaturing agents such as SDS or urea are present in the reaction buffer. On the other hand, calcium ions protect proteinase K against autoproteolysis.

These enzymes have the ability to catalyse the hydrolysis of peptide bonds, at the same time exhibiting a broad substrate spectrum, therefore they are mainly used in DNA isolation processes and other applications of molecular biology, as well as in the removal of protein contaminants from non-protein materials.

Proteinase K is an enzyme known for a long time, isolated from the post-culture fluid of Tritirachium album mould. However, the production process is long and is characterised by low production efficiency per 1 litre of post-culture fluid. Attempts have also been made to construct bacterial expression systems enabling the production of proteinase K in the form of inclusion bodies, but the problem of protein renaturation prevented the production of significant amounts of the enzyme (Gunkel, F. A. and Gassen, H. G. (1989) Eur. J. Biochem. Vol. 179 (1), 185-194; Samal, B. B. et al. (1996) Adv. Exp. Med. Biol. Vol. 379, 95-104).

Further, in patent EP1360283 a method of producing proteinase K in yeast systems in the form of a translational fusion of a zymogen with the signal peptide of the Saccharomyces cerevisiae alpha-factor was described. In this case, the autocatalytic activation of the zymogen to the active form of the protein occurs during secretion or in the post-culture fluid, but it can also occur prematurely and is therefore not a strictly controlled process.

Patent PL 213045 describes a mutant of the Tritirachium album proteinase K zymogen, containing an amino acid sequence recognised by the Kex2 protease placed between the propeptide and the protein sequence with the activity of mature proteinase K. Such modification of the zymogen (or enzymatically inactive form of the protein) leading to the controlled cleavage of the propeptide (or the portion of the protein acting as the internal chaperone) in yeast cells and the production of the mature form of proteinase K or a mutant thereof (i.e. the protein with the activity of mature Proteinase K) enables efficient production of the enzyme in the yeast host, in particular in cells of the recombinant Pichia pastoris strain.

Despite the known solutions, the search for new, efficient systems allowing for the effective and cheap biosynthesis of enzymes from the K proteinase family is continued. Work on the construction of a system that allows for the biosynthesis and easy isolation of an enzyme with proteinase K activity has been underway for numerous years. The object of the invention is to provide such a system allowing for the efficient production of a protein with proteinase K activity and new proteinase K mutants that can be used to obtain such a desired system.

Another object of the invention is to obtain a new protein with a proteolytic activity similar to that of proteinase K, which, however, can be easily inactivated in order to avoid the undesirable proteolytic activity of the reaction mixture obtained after the end of the proteolytic reaction. In case proteinase K is used as a reagent to remove all proteins, e.g. during nucleic acid purification, it is undesirable to leave enzyme contamination with proteinase activity in the end product. Therefore, the production of a protein that can be inactivated more easily than wild-type proteinase K is highly desirable.

Unexpectedly, the above-mentioned complex technical effect has been achieved with the present invention.

Unexpectedly, the object defined above was achieved with the present invention.

The invention relates to a mutant of the Tritirachium album proteinase K zymogen containing a mutation that increases the efficiency of expression in yeast cells and facilitates the inactivation of mature proteinase K under stress at the C-terminus, the mutation being an additional Gly residue at the C-terminus.

Preferably, the Tritirachium album proteinase K zymogen mutant of the invention has the sequence shown as sequence No. 9.

Unexpectedly, the modification of the zymogen proposed by the invention increases the enzyme production efficiency in yeast cells, especially of Pichia pastoris according to the described Example 9.

The invention also relates to a mutant of the Tritirachium album mature proteinase K containing a mutation that increases the efficiency of expression in yeast cells and facilitates the inactivation of mature proteinase K under stress at the C-terminus, the mutation being an additional Gly residue at the C-terminus. Preferably it has the sequence shown as sequence No. 22.

The invention also relates to a DNA sequence containing the coding sequence of a proteinase K zymogen mutant according to the invention as defined above or a fragment thereof. Preferably, it has sequence No. 8.

The invention also relates to the sequence of oligonucleotides with the symbols ProtK-G-F and ProtK-G-R for the production of a DNA sequence encoding a mutant proteinase K zymogen according to the invention as defined above, or a fragment thereof selected from the sequences No. 6 and 7.

The invention also relates to an expression plasmid containing a DNA sequence encoding a mutant proteinase K zymogen according to the invention as defined above, as described in Example 4. Preferably, the plasmid according to the invention has a sequence selected from the following sequences: sequence No. 16.

The invention also relates to a recombinant yeast strain transformed with any plasmid according to the invention as defined above, as described in Example 6. Preferably, the yeast strain according to the invention may be obtained from one of the known yeast strains selected from Pichia sp., Hansenula sp., Saccharomyces sp., Schizosaccharomyces sp., Yarrovia sp. or Kluyveromyces sp., preferably selected from Pichia pastoris strains.

The invention also relates to a method for obtaining a protein with Tritirachium album proteinase K activity, characterised in that yeast cells transformed with an expression plasmid encoding a mutant of the Tritirachium album proteinase K zymogen containing at the C-terminus a mutation increasing the efficiency of expression in yeast cells and facilitating inactivation of mature proteinase K under stress conditions are cultured, wherein the mutation is an additional Gly residue located at the C-terminus, and then the obtained protein is isolated and purified from the post-culture fluid.

Preferably, yeast cells according to the invention as defined above are cultured, preferably at a temperature of about 28 to 30° C., preferably on a medium containing glycerol or methanol as the carbon source, the expression of the modified proteinase K gene being induced with methanol. Equally preferably, the protein with the activity of Tritirachium album proteinase K is isolated from the post-culture fluid by hydrophobic interaction chromatography or by chromatography on a hydroxyapatite bed, and then purified by dialysis against ammonium carbonate solution and subsequently freeze-dried as described in Example 9.

An expression system allowing the efficient biosynthesis of the mature form of the modified Tritirachium album ATCC 22625 proteinase K in Pichia pastoris cells was constructed. The gene encoding the modified protein zymogen shown in sequence No. 8 was amplified by PCR on the template sequence of the gene encoding the wild-type version of the protein shown in sequence No. 5 as described in Example 2 and sequenced. From the amplified sequence, the nucleotide sequence encoding the signal peptide shown in sequence 2 was removed, and the DNA sequence encoding the wild-type protein propeptide was left, according to the sequence No. 3, stabilising the proteinase K zymogen against premature activation to the mature form of an enzymatically active protein (Wolfgang Ebeling, et al, Proteinase K from Tritirachium album Limber, Eur. J Biochem. 47, 91-97, 1974). Additionally, an amino acid residue of glycine was added at the C-terminus of the mature protein, which, unexpectedly, increased the autolysis efficiency of the mature form of the mutant protein under stress conditions, at an increased concentration of a surfactant. Such a modification of the wild-type protein allowed for the efficient inactivation of the enzyme under appropriate buffer conditions as described in Example 10, which is a particularly advantageous characteristic allowing for easy inactivation of the enzyme in nucleic acid purification processes where high quality and purity of DNA and RNA products free of protein impurities are required, without the use of additional chemical or physical factors that can damage the isolated preparations of nucleic acids.

The Pichia pastoris/pD912-ProtK-G expression strain obtained in the invention, as described in Example 6, allows for the production of significant amounts of functional modified Tritirachium album proteinase K, and also allows for the production of a modified enzyme that does not differ in activity from the enzyme isolated from the wild-type strain. The obtained mature proteinase K mutant protein, shown in sequence 22, does not significantly differ in activity from the protein isolated from Tritirachium album despite the presence of an additional glycine amino acid residue at the C-terminus of the mature protein.

An embodiment of the invention is also the DNA sequence of the expression plasmid with the symbol pD912-ProtK and sequence 21 obtained as a result of DNA amplification, as described in Example 7, using the primers with sequence 18 and the symbol ProtK-G-out-F and sequence 19 and the symbol ProtK-G-out-R, which contains a DNA fragment with sequence 20, encoding an amino acid sequence identical to the mutant version of proteinase K but lacking an additional glycine amino acid residue at the C-terminus of the mature protein, according to sequence 17.

An embodiment of the invention is the recombinant Pichia pastoris strain with the symbol Pichia pastoris/pD912-ProtK as described in Example 8, which is constituted by Pichia pastoris BG10 yeast cells containing the gene encoding the modified Tritirachium album proteinase K shown in sequence 20.

An embodiment of the invention is also the DNA sequence of the expression plasmid with the symbol pPink_HC-ProtK-WT and sequence 11 obtained by de novo DNA synthesis and cloning into the vector with the symbol pPink_HC and sequence 10 as described in Example 1. The obtained pPink_HC-ProtK-WT expression plasmid contains a DNA fragment with sequence 5 and the symbol ProtK-WT encoding the amino acid sequence of the wild-type version of Tritirachium album proteinase K. It contains the native proteinase K signal peptide (sequence 2) together with the native propeptide (sequence 3).

An embodiment of the invention is the recombinant Pichia pastoris strain with the symbol Pichia Pink/pPink_HC-ProtK-WT as described in Example 5, which is constituted by Pichia Pink yeast cells (ThermoFisher, cat. no. A11150) containing the gene encoding the wild-type version of Tritirachium album proteinase K shown in sequence 1.

An embodiment of the invention is also a method for the production of modified Tritirachium album proteinase K as described in Example 9, consisting in the extracellular production of the enzyme by cells of a recombinant yeast strain in liquid media enriched with calcium ions (Ca²⁺) characterised in that cells of the yeast strain with the symbol Pichia Pink/pPink_HC-ProtK-WT or Pichia pastoris/pD912-ProtK or Pichia pastoris/pD912-ProtK-G are cultured at 28-30° C., the expression of the gene encoding wild-type or modified proteinase K is induced with methanol, and then the protein is isolated and purified from the post-culture fluid by hydrophobic interaction chromatography, and finally the purified enzyme is freeze-dried. A method of chromatographic purification on a hydroxyapatite bed can also be used here.

By using the invention, an expression system is obtained that enables efficient production of the modified Tritirachium album proteinase K (ProtK-G) in recombinant Pichia pastoris/pD912-ProtK-G yeast cells, which is about 10% more efficient compared to the expression level of the native proteinase (ProtK) performed under the same conditions in the same expression system in the recombinant Pichia pastoris/pD912-ProtK strain and as much as approximately 30% more efficient compared to expression in the wild-type proteinase K in the Pichia Pink/pPink_HC-ProtK-WT strain. The Pichia Pink™ strain is a commercial expression system offered by Thermo Fisher, USA for high-efficiency production of recombinant proteins in the Pichia Pink yeast system.

As a result of the invention, the Pichia pastoris/pD912-ProtK-G expression strain was obtained, which resulted to be the most efficient in the production of Tritirachium album proteinase K in the recombinant system among all the systems tested during the implementation of the invention. With a 10% yield difference and provided the process is scaled up to a 1000 L production scale, it is thus possible to produce over 260 g more protein per process.

### DESCRIPTION OF FIGURES AND SEQUENCES

FIG. 1—shows a structure of an expression plasmid with sequence 11 and the symbol pPink_HC-ProtK-WT encoding the wild-type version of proteinase K.

FIG. 2—shows a structure of an expression plasmid with sequence 16 (or 21) and the symbol pD912-ProtK-G (or pD912-ProtK) encoding the recombinant version of proteinase K with the mutated C-terminus (or the wild-type version of the mature protein).

FIG. 3—Comparison of Tritirachium album proteinase K expression efficiency in three expression strains: Pichia Pink/pPink_HC-ProtK-WT, Pichia pastoris/pD912-ProtK-G and Pichia pastoris/pD912-ProtK.

FIG. 4—Comparison of the protein stability of the recombinant version of ProtK-G and native ProtK_WT under conditions typical for nucleic acid purification and stress conditions, with an amount of surfactant increased up to 1%. The stability of the recombinant version of ProtK-G and the native ProtK WT after 30 min. of incubation under conditions typical for DNA and/or RNA isolation (left panel) or under stress conditions with increased surfactant concentration (middle panel) was shown on SDS-PAGE gels. On the right panel both proteins after an additional 30 min. incubation under stress conditions were shown. Both proteins, ProtK-G and ProtK-WT, do not undergo autolysis under conditions typical for nucleic acid isolation and remain at a similar level, while under stress conditions, the recombinant ProtK-G protein is degraded faster and more efficiently compared to the wild-type version, ProtK-WT.

FIG. 5—Comparison of Tritirachium album proteinase K expression efficiency in three expression strains: Pichia Pink/pPink_HC-ProtK-WT, Pichia pastoris/pD912-ProtK-G and Pichia pastoris/pD912-ProtK. The yeast fermentation was carried out using the same protocol under the same conditions for all expression systems. After the culture was finished, the amount of cell biomass in the post-fermentation culture after the separation of the fermentation medium by centrifugation, presented in grams of the sediment per 1 litre of culture was measured. The content of proteinase K in the post-fermentation medium after the separation of the cells was measured by densitometry and by HPLC. The relative performance of the three expression systems was compared with the most efficient system, Pichia pastoris/pD912-ProtK-G assumed as 100%.

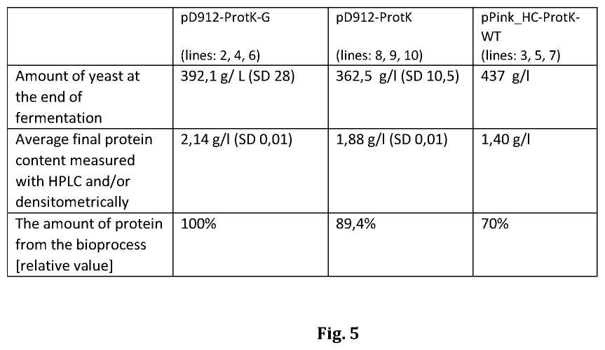

Sequence 1—shows the amino acid sequence of the Tritirachium album proteinase K wild-type protein with the symbol ProtK-WT

Sequence 2—shows the amino acid sequence of the signal peptide of the Tritirachium album proteinase K wild-type protein with the symbol ProtK-WT SP

Sequence 3—shows the amino acid sequence of the propeptide of the Tritirachium album proteinase K wild-type protein with the symbol ProtK-WT propeptide

Sequence 4—shows the amino acid sequence of the fragment of the enzymatically active protein of the Tritirachium album mature proteinase K with the symbol ProtK-WT mature

Sequence 5—shows the nucleotide sequence of the 1173 nucleotide-long gene encoding the Tritirachium album wild-type proteinase K protein version with the symbol ProtK-WT

Sequence 6—shows the sequence of the 41 nucleotide-long oligonucleotide with the symbol ProtK-G-F used in the PCR reaction to amplify sequence 8

Sequence 7—shows the sequence of the 41 nucleotide-long oligonucleotide with the symbol ProtK-G-R used in the PCR reaction to amplify sequence 8

Sequence 8—shows the nucleotide sequence of the 1139 nucleotide-long gene encoding the fragment of the propeptide and peptide of the mature protein of the recombinant version of the Tritirachium album proteinase K with the symbol pro-ProtK-G

Sequence 9—shows the 370 amino acid-long sequence of the propeptide and peptide of the enzymatically active protein fragment of the K Tritirachium album mature recombinant proteinase K protein with the symbol pro-ProtK-G

Sequence 10—Shows the nucleotide sequence of the 7667 nucleotide-long expression vector pPink_HC purchased from Thermo Fisher, USA, Cat. No. A11152 containing the methanol inducible Saccharomyces cerevisiae AOX1 promoter

Sequence 11—shows the 8805 nucleotides-long nucleotide sequence of the pPink_HC-ProtK-WT expression vector with the symbol pPink_HC-ProtK-WT containing sequence 5 under the control of the methanol inducible Saccharomyces cerevisiae AOX1 promoter

Sequence 12—shows the amino acid sequence of the Tritirachium album proteinase K recombinant version protein with the symbol ProtK-G and the length of 460 amino acids with the modified C-terminus of the protein

Sequence 13—shows the 90 amino acid-long sequence of the signal peptide of the Saccharomyces cerevisiae proteinase alpha factor protein with the symbol ProtK-G SP

Sequence 14—shows the nucleotide sequence of the 1324 nucleotide-long gene encoding the Tritirachium album proteinase K recombinant version protein with the symbol ProtK-G

Sequence 15—Shows the nucleotide sequence of the 3837 nucleotide-long expression vector pD912 purchased from ATUM, USA, Cat. No. 867858d1 containing the methanol inducible Saccharomyces cerevisiae AOX1 promoter

Sequence 16—shows the 4928 nucleotide-long nucleotide sequence of the pD912-ProtK-G expression vector with the symbol pD912-ProtK-G containing sequence 14 under the control of the methanol inducible Saccharomyces cerevisiae AOX1 promoter

Sequence 17—shows the amino acid sequence of the Tritirachium album proteinase K recombinant version protein with the symbol ProtK and the length of 459 amino acids with the native C-terminus of the protein (without the added amino acid glycine at position 460)

Sequence 18—shows the sequence of the 59 nucleotide-long oligonucleotide with the symbol ProtK-G-out-F used in the PCR reaction to amplify sequence 20 Sequence 19-shows the sequence of the 36 nucleotide-long oligonucleotide with the symbol ProtK-out-G-used in the PCR reaction to amplify sequence 20

Sequence 20—shows the nucleotide sequence of the 1324 nucleotide-long gene encoding the Tritirachium album proteinase K recombinant version protein with the symbol ProtK (G460stop)

Sequence 21—shows the 4928 nucleotide-long nucleotide sequence of the pD912-ProtK expression vector with the symbol pD912-ProtK containing sequence 20 under the control of the methanol inducible Saccharomyces cerevisiae AOX1 promoter

Sequence 22—shows the amino acid sequence of the fragment of the enzymatically active protein fragment of the Tritirachium album mature proteinase K with the symbol ProtK-WT mature

Example 1. Obtaining the DNA Sequence of the Gene Encoding the Tritirachium album Wild-Type Proteinase K with Sequence 1 and the Symbol ProtK-WT.

In order to obtain the DNA of the gene encoding the wild-type proteinase K protein with sequence 1 and the symbol ProtK-WT containing the signal peptide with sequence 2 and the symbol ProtK-WT SP, the propeptide with sequence 3 and the symbol ProtK-WT and the mature enzymatically active proteinase K polypeptide with sequence 4 and the symbol ProtK-WT mature, the DNA sequence based on the proteinase K protein sequence available in the UniProtKB database number P06873 (https://www.uniprot.org/uniprot/P06873) was designed. The DNA sequence encoding the wild-type proteinase K was obtained by reverse translation of the polypeptide sequence No. 1 with the symbol ProtK-WT. The designed DNA sequence was optimised for efficient expression in Pichia pastoris yeast. In addition, to allow cloning into an expression vector, recognition sites for restriction enzymes EcoRI and KpnI were designed at the 5- and 3-ends, respectively. The de novo gene synthesis was commissioned to GeneART (https://www.thermofisher.com/pl/en/home/life-science/cloning/gene-synthesis/geneart-gene-synthesis.html). The DNA sequence of the obtained gene with the symbol ProtK-WT, is shown as sequence 5.

Example 2. Obtaining the DNA Sequence of the Gene Encoding a Fragment of Tritirachium album Recombinant Proteinase K with the Glycine Amino Acid Attached to the C-Terminus of the Protein with Sequence 8 and the Symbol Pro-ProtK-G.

In order to obtain a DNA fragment of the gene with sequence 8 and the symbol pro-ProtK-G encoding a fragment of the recombinant Tritirachium album proteinase K with the glycine amino acid attached to the C-terminus of the protein with sequence 9 and the symbol pro-ProtK-G, the DNA fragment obtained in Example 1 with sequence 5 and the symbol ProtK-WT is amplified by PCR using two oligonucleotide primers with sequence 6 and the symbol ProtK-GF and sequence 7 and the symbol ProtK-GR using an amplification kit purchased from New England Biolabs, Inc., Cat. No. 50-995-156 under the conditions recommended by the manufacturer. In addition, to enable the cloning of the DNA fragment of sequence 8 and the pro-ProtK-G symbol into an expression vector, the oligonucleotides used in the PCR reaction contain a restriction enzyme SapI recognition site. The obtained DNA fragment with sequence 8 and the symbol pro-ProtK-G, is precipitated with ethanol and used for cloning into an expression vector.

Example 3. Obtaining an Expression Plasmid with Sequence 11 and the Symbol pPink_HC-ProtK-WT Containing the Gene Encoding the Polypeptide Sequence of the Tritirachium album Wild-Type Proteinase K with Sequence 1 and the Symbol ProtK-WT.

In order to obtain the expression plasmid with sequence 11 and the symbol pPink_HC-ProtK-WT, the DNA fragment purified by ethanol precipitation with sequence 1 and the symbol ProtK-WT, obtained in Example 1, is digested with EcoRI and KpnI restriction enzymes, and then ligated with the DNA of the plasmid vector with sequence 10 and the symbol pPink HC purchased from Thermo Fisher, USA, Cat. No. A11152 digested with the same restriction enzymes.

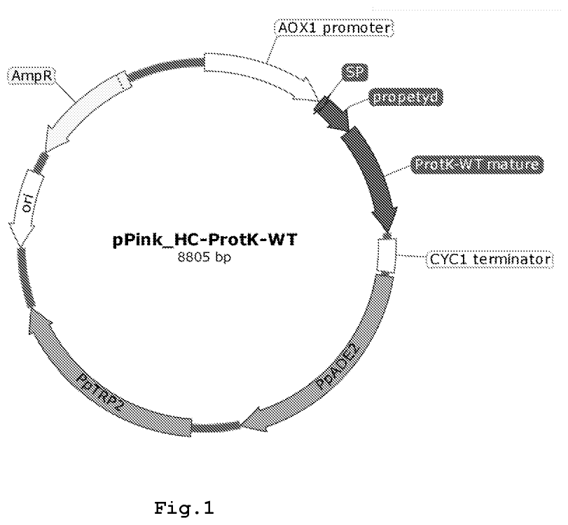

The ligation mixture is transformed into Escherichia coli TOP10F competent cells and plated in Petri dishes with LA medium (1% K-peptone; 0.5% yeast extract; 1% NaCl; 1.5% agar) containing 100 μg/ml ampicillin. As a result of the isolation of plasmid DNA from the obtained bacterial colonies, an expression plasmid with sequence 11 and the symbol pPink_HC-ProtK-WT is obtained, containing the gene with sequence 5 and the symbol ProtK-WT, encoding the Tritirachium album proteinase K wild-type version with sequence 1 and the symbol ProtK-WT. The structure of the plasmid with sequence 11 and the symbol pPink_HC-ProtK-WT is shown in FIG. 1.

Example 4. Obtaining an Expression Plasmid with Sequence 16 and the Symbol pD912-ProtK-G Containing the Gene of Sequence 14 and the Symbol ProtK-G Encoding the Polypeptide Sequence of the Tritirachium album Recombinant Proteinase K with Sequence 12 and the Symbol ProtK-G.

The DNA fragment of sequence 8 and the symbol pro-ProtK-G obtained in Example 2 is digested with the SapI restriction enzyme, and then ligated with the DNA of the plasmid vector with sequence 15 and the symbol pD912 purchased from ATUM, Newark, USA, Cat. No. 867858d1 digested with the same restriction enzyme. As a result of the ligation of the DNA fragment with sequence 8 and the symbol pro-ProtK-G with the DNA of the pD912 vector with sequence 15, sequence 8 with the symbol pro-ProtK-G is joined in the correct reading frame (translational fusion) with the signal peptide on the pD912 vector with sequence 13 and the symbol ProtK-G SP.

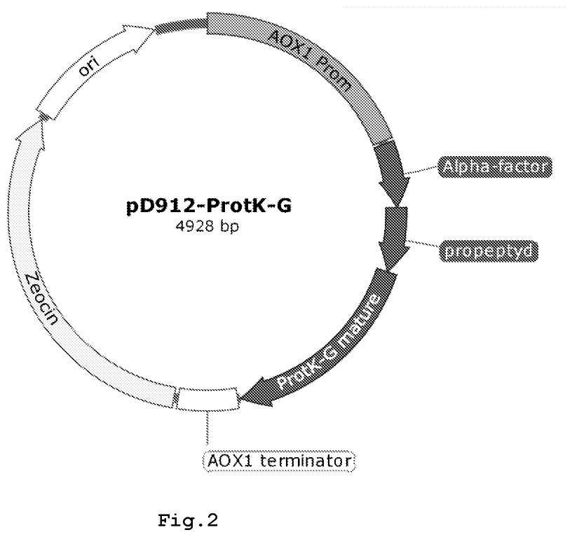

The ligation mixture is transformed into Escherichia coli TOP10F competent cells and plated in Petri dishes with LA medium (1% K-peptone; 0.5% yeast extract; 1% NaCl; 1.5% agar) containing 25 μg/ml zeocin. As a result of the isolation of plasmid DNA from the obtained bacterial colonies, an expression plasmid with sequence 16 and the symbol pD912-ProtK-G is obtained, containing the gene with sequence 14 and the symbol ProtK-G, encoding the Tritirachium album recombinant proteinase K with sequence 16 and the symbol pD912-ProtK-G shown in FIG. 2.

Example 5. Obtaining a Recombinant Pichia pastoris Strain with the Symbol Pichia Pink/pPink_HC-ProtK-WT.

In order to obtain the recombinant Pichia Pink/pPink_HC-ProtK-WT strain, the Pichia Pink yeast cells are transformed with the linear form of the expression plasmid DNA with sequence 11 and the symbol pPink_HC-ProtK-WT obtained by previous digestion of the plasmid DNA with sequence 11 and the symbol pPink_HC-ProtK-WT with the SpeI restriction enzyme (Thermo Fisher, Cat. No. ER1251). Selection of positive Pichia Pink yeast cell clones is performed on PAD selection medium as recommended by the Pichia Pink cell supplier (ThermoFisher, Cat. No. A11150, manual version MAN0000717). The presence of the recombinant ProtK-WT gene embedded to the genome of the yeast with the symbol Pichia Pink/pPink_HC-ProtK-WT is confirmed by PCR amplification using oligonucleotides as in Example 2 on a template of genomic DNA isolated from yeast cells.

Example 6. Obtaining a Recombinant Pichia pastoris Strain with the Symbol Pichia pastoris/pD912-ProtK-G

In order to obtain the recombinant Pichia pastoris/pD912-ProtK-G strain, a transformation of the yeast Pichia pastoris strain BG10 (ATUM, US Cat. No. PPS-9010) with the linear form of the expression plasmid DNA with sequence 16 and the symbol pD912-ProtK-G obtained by previous digestion of the plasmid DNA with sequence 16 and the symbol pD912-ProtK-G with the PmeI restriction enzyme (Thermo Fisher, Cat. No. ER1342) is performed. Selection of positive clones of Pichia pastoris/pD912-ProtK-G yeast cells is performed on YPDS selection medium supplemented with 200 μg/ml zeocin and subsequently they are transferred to the YPD medium supplemented with 200 μg/ml zeocin, according to the recommendations of the Pichia pastoris strain BG10 cell supplier (ATUM, USA). The presence of the recombinant ProtK-G gene embedded to the genome of the yeast with the symbol Pichia pastoris/pD912-ProtK-G is confirmed by PCR amplification using oligonucleotides as in Example 2 on a template of genomic DNA isolated from yeast cells.

Example 7. Obtaining an Expression Plasmid with Sequence 21 and the Symbol pD912-ProtK Containing the Gene of Sequence 20 and the Symbol ProtK Encoding the Polypeptide Sequence of the Tritirachium album Recombinant Proteinase K with Sequence 17 and the Symbol ProtK.

The expression plasmid with sequence 16 and the symbol pD912-ProtK-G containing the gene with sequence 14 and the symbol ProtK-G encoding the polypeptide sequence of the recombinant Tritirachium album proteinase K with sequence 12 and the symbol ProtK-G obtained according to Example 4 is used as a template in the PCR process to obtain an expression vector with sequence 21 and the symbol pD912-ProtK containing the gene with sequence 20 and the symbol ProtK encoding the polypeptide sequence of the Tritirachium album recombinant proteinase K with sequence 17 and the symbol ProtK. In the PCR process, two oligonucleotides are applied as primers, one with sequence 18 and the symbol ProtK-G-out-F and the other with sequence 19 and the symbol ProtK-G-out-R using an amplification kit purchased from New England Biolabs, Inc., Cat. No. 50-995-156, under the conditions recommended by the manufacturer.

The PCR mixture is incubated with the DpnI restriction enzyme (Thermo Fisher, Cat. No. ER1701), transformed into Escherichia coli DH5 (alpha) competent cells and plated in Petri dishes with LA medium (1% K-peptone; 0.5% yeast extract; 1% NaCl; 1.5% agar) containing 25 μg/ml zeocin. As a result of the isolation of plasmid DNA from the obtained bacterial colonies, an expression plasmid with sequence 21 and the symbol pD912-ProtK is obtained, containing the gene with sequence 20 and the symbol ProtK, encoding the recombinant Tritirachium album proteinase K with sequence 17 and the symbol ProtK. The structure of the plasmid with sequence 21 and the symbol pD912-ProtK is shown in FIG. 2.

Example 8. Obtaining a Recombinant Pichia pastoris Strain with the Symbol Pichia pastoris/pD912-ProtK

In order to obtain the recombinant Pichia pastoris/pD912-ProtK strain, a transformation of the yeast Pichia pastoris strain BG10 (ATUM, US Cat. No. PPS-9010) with the linear form of the expression plasmid DNA with sequence 21 and the symbol pD912-ProtK obtained by previous digestion of the plasmid DNA with sequence 21 and the symbol pD912-ProtK with the PmeI restriction enzyme (Thermo Fisher, Cat. No. ER1342) is performed. Selection of positive clones of Pichia pastoris/pD912-ProtK yeast cells is performed on YPDS selection medium supplemented with 200 μg/ml zeocin and subsequently they are transferred to the YPD medium supplemented with 200 μg/ml zeocin, according to the recommendations of the Pichia pastoris strain BG10 cell supplier (ATUM, USA). The presence of the recombinant ProtK gene embedded to the genome of the yeast with the symbol Pichia pastoris/pD912-ProtK is confirmed by PCR amplification using oligonucleotides as in Example 2 on a template of genomic DNA isolated from yeast cells.

Example 9. Obtaining Tritirachium album Proteinase K and Comparing the Expression Efficiency of the Recombinant (ProtK-G) and Wild-Type (ProtK-WT or ProtK) Version of Proteinase K Using Pichia pastoris/pD912-ProtK-G, Pichia Pink/pPink_HC-ProtK-WT Cells or Pichia pastoris/pD912-ProtK Cells, Respectively.

The expression strains of Pichia Pink/pPink_HC-ProtK-WT, Pichia pastoris/pD912-ProtK-G or Pichia pastoris/pD912-ProtK, obtained in Examples 5, 6 or 8, respectively, producing the recombinant (ProtK-G), native (ProtK-WT) or wild-type version of the mature proteinase K polypeptide, respectively, are grown on MGY medium for 18-20 hours at 30° C. until the cell density measured by a spectrophotometer at 600 nm, OD 600 is higher than 2. Subsequently, BSM medium in a steel reactor with a working volume of 20 litres, containing 2% glycerol as carbon source is inoculated with the obtained material, and the culture is conducted at 30° C. for 46-48 hours while glycerol as carbon source is added. After the appropriate cell density is achieved, the culture is induced to express the modified or native protein K gene by addition of methanol. The culture is continued for another 3-4 days (72-96 h), while adding methanol as a carbon source and inducer of gene expression. At the end of the culture and from samples collected during the culture, yeast cells are separated from the post-culture fluid by centrifugation. The content of proteinase K in the post-fermentation medium, after the cells are separated, in the post-culture medium is analysed by SDS-PAGE and/or by HPLC.

Protein from the post-culture medium, after separation of the yeast biomass by centrifugation or by means of tangential flow filtration using a filter cassette with pores of 0.2 micrometers, is purified by hydrophobic interaction chromatography using a bed, e.g. Phenyl Sepharose HS (Cytiva Cat. No. 17097399) or by chromatography on a hydroxyapatite bed, followed by purification by dialysis against ammonium carbonate solution, followed by freeze-drying to obtain an enzymatically active final product.

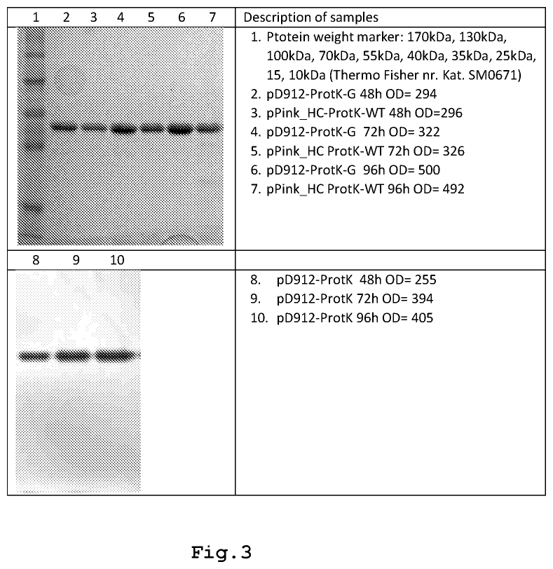

The comparison of expression levels during the culture, and 2, 3 and 4 days after the induction in Pichia Pink/pPink_HC-ProtK-WT, Pichia pastoris/pD912-ProtK-G and Pichia pastoris/pD912-ProtK strains is shown in FIG. 3. The efficiency of recombinant proteinase K (ProtK-G) expression in the Pichia pastoris/pD912-ProtK-G strain is approximately 10% higher compared to the expression level of the native proteinase (ProtK) in the same expression system in the Pichia pastoris/pD912-ProtK strain and as much as approximately 30% higher compared to the expression in the Pichia Pink/pPink_HC-ProtK-WT strain. The Pichia Pink™ strain is a commercial expression system offered by Thermo Fisher, USA, optimised for high-efficiency production of recombinant proteins in the Pichia Pink yeast system. Despite the use of an optimised expression host, protein production in the native sequence was 30% lower compared to wild-type Pichia pastoris cells using the same production protocol.

The Pichia pastoris/pD912-ProtK-G expression strain proved to be the most efficient for the production of Tritirachium album proteinase K. With a 10% yield difference and provided the process is scaled up to a 1000 L production scale, it is thus possible to produce 260 g more protein in one fermentation.

Example 10. Possibility of Faster Inactivation and Removal of Proteinase K with a Modified Sequence, ProtK-G in Comparison to the Native Proteinase K, ProtK-WT in the Process of Nucleic Acid Purification.

Proteinase K from Tritirachium album, as a broad-spectrum endopeptidase with very high specific activity, is widely used in the isolation of nucleic acids (DNA and RNA) with no damage to their structure, for protein digestion, including DNases and RNases and other nucleic acid-interacting proteins, which may reduce the purity of the final DNA or RNA products. Proteinase K retains high activity under a wide spectrum of buffer conditions and is additionally activated by the addition of a small amount of SDS surfactant, in the range 0.1-0.5%. Effective digestion of protein contaminants, including proteinase K added in the isolation process, is a condition for high-quality DNA or RNA products, and any protein residues present during the purification process negatively affect the quality of the purified samples. It is extremely important in preparing samples used for sequencing and diagnostics using qPCR, where protein contamination in the prepared samples may cause false results.

In order to demonstrate the possibility of efficient inactivation and removal of proteinase K in the DNA and/or RNA purification process in the case of the proteinase K variant of the present invention, the activity of both variants (ProtK-G with sequence No. 12 and ProtK-WT with sequence No. 1) was compared under the conditions typical for the purification of nucleic acids (incubation in 10 mM Tris-HCl pH7.5, 37° C.) without a stress factor and under stress conditions, with the addition of 1% SDS (incubation in 10 mM Tris-HCl pH7.5, 1% SDS, 37° C.).

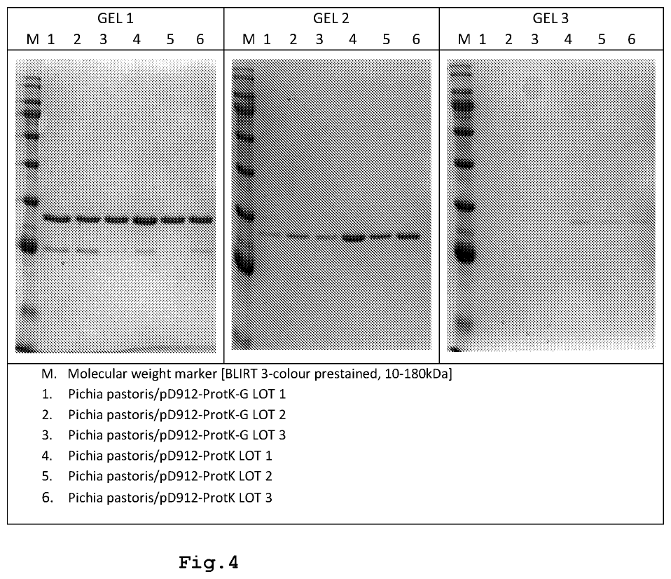

Unexpectedly, it was found that the recombinant version of proteinase K or ProtK-G loses activity at a faster rate under stressful conditions compared to the wild-type version or ProtK-WT, which allows for easier removal of the residual proteinase K after the nucleic acid-interacting protein digestion step during DNA and/or RNA isolation, in contrast to the native protein, ProtK-WT. The results of the comparison of both proteins under standard and stress incubation conditions are presented in FIG. 4.

The digestibility shown by the mutant after incubation under stress conditions remains at only 1.9%, compared to 69.7% of the original activity. The reference protein, ProtK-WT, maintains more than 30% digestibility when incubated under the same conditions with a surfactant.

The obtained results suggest that under appropriate conditions (1% surfactant) it is possible to almost completely inactivate the enzyme in the mutant variant, ProtK-G, of the present application, without the use of a dedicated reagent/inhibitor [Pefabloc® SC] or a drastic increase in temperature, as in the case of other recombinant proteinases K and commercially available products known in the prior art from patent applications EP3670655A1 [New England Biolabs Thermolabile Proteinase K] or WO2019170809A1 [Arctic Zymes Proteinase]. The addition of 1% ionic surfactant (surfactant, sodium lauryl sulphate-SDS) and 30-minute incubation at an elevated temperature of 37° C., causes a decrease in the activity of the mutant proteinase K, ProtK-G according to the present invention, almost to zero, in contrast to the native variant, ProtK-WT.

This provides a possibility to easily inactivate the enzyme, especially in nucleic acid purification processes, where high purity of DNA and RNA products is required, without the use of additional chemical or physical factors that can damage the isolated nucleic acid products.

Example 11. Evaluation of the Thermal Unfolding of the Recombinant (ProtK-G) and Wild-Type (ProtK-WT) Proteinase K in Harsh Conditions Buffering Using the nanoDSF Technology.

The nanoDSF technology was used to evaluate the thermostability of the recombinant variant of proteinase K with C-terminal modification with glycine (Sequence 22) and wild type of the enzyme (Sequence 4; marketed product known from the prior art). Both proteases were prepared at the same concentration in the ATL buffer (QIAGEN, cat. No. 19076) comprising an ionic surfactant or the ACL buffer (QIAGEN, cat. No. 939017) comprising a chaotropic guanidinium salt at pH>8, respectively. The thermal stability measurements were performed using a differential scanning fluorescence instrument, Prometheus. Samples were heated from 20 to 95 degrees Celsius and changes in intrinsic fluorescence were recorded at 330 and 350 nm. The data was analyzed for determining protein thermal unfolding using the Prometheus software. Melting temperature (Tm) for ProtK-G and ProtK-WT were calculated from the first derivative of 330/350 nm florescence and are presented in Table 1.

Unexpectedly, it was found that the recombinant version of proteinase K, ProtK-G, has significantly lower melting temperature in buffer supplemented with ionic surfactant or chaotropic guanidinium salt (Tm=54.5° C. and 40.1° C., respectively) compared to wild-type enzyme, ProtK-WT (Tm=61.0° C. and 47.0° C., respectively) (Table 1). The obtained results are in accordance with the data observed in the Example no. 10, where ProtK-G loses activity at a faster rate than ProtK-WT in the presence of ionic surfactant.

The increased thermolability of ProtK-G variant of present invention over the wild type enzyme (ProtK-WT) provides possibility for easier inactivation and removal of enzyme during the standard nucleic acid purification processes at lower temperature.

TABLE 1
Comparison of the thermal unfolding
of the recombinant version of ProtK-G and
native ProtK-WT using the nanoDSF technology.

| | Melting temperature (Tm) | |
|---|---|---|
| | ProtK-G | ProtK-WT |
| Buffer ATL | 54.5° C. | 61.0° C. |
| (ionic surfactnat) | | |
| Buffer ACL | 40.1° C. | 47.0° C. |
| (chaotropic | | |
| guanidinium salt) | | |

1-14. (canceled)

15. A mutant of the Tritirachium album proteinase K zymogen containing a mutation at the C-terminus that increases the efficiency of expression in yeast cells and facilitates the inactivation of mature proteinase K under stress, the mutation being an additional Gly residue at the C-terminus.

16. The mutant according to claim 15, wherein the mutant possesses a sequence presented as SEQ ID NO. 12.

17. A mutant of the Tritirachium album mature proteinase K containing a mutation at the C-terminus that increases the efficiency of expression in yeast cells and facilitates the inactivation of mature proteinase K under stress, the mutation being an additional Gly residue at the C-terminus.

18. The mutant according to claim 18, wherein the mutant possesses a sequence presented as SEQ ID NO. 22.

19. A DNA sequence comprising the coding sequence of the proteinase K zymogen mutant according to claim 15 or its fragment.

20. The DNA sequence according to claim 19, wherein the DNA sequence possesses a sequence presented as SEQ ID NO. 8.

21. An oligonucleotide sequence for obtaining a DNA sequence encoding a mutant of the proteinase K zymogen according to claim 15, or a fragment thereof selected from SEQ ID NOS. 6 and 7.

22. An expression plasmid comprising a DNA sequence encoding a mutant of the proteinase K zymogen according to claim 15.

23. The plasmid according to claim 22, wherein the plasmid possesses a sequence selected from SEQ ID NO. 16.

24. A recombinant yeast strain transformed with a plasmid according to claim 22.

25. A recombinant yeast strain transformed with a plasmid according to claim 23.

26. The recombinant yeast strain of claim 24, wherein the recombinant yeast strain has been obtained from one selected from the group consisting of Pichia sp., Hansenula sp., Saccharomyces sp., Schizosaccharomyces sp., Yarrovia sp and Kluyveromyces sp.

27. A method for obtaining a protein with Tritirachium album proteinase K activity, wherein yeast cells transformed with an expression plasmid encoding a mutant of the Tritirachium album proteinase K zymogen containing a mutation at the C-terminus, increasing the efficiency of expression in yeast cells and facilitating inactivation of mature proteinase K under stress conditions are cultured in a culture fluid, wherein the mutation is an additional Gly residue located at the C-terminus, and the obtained protein is isolated and purified from the culture fluid.

28. The method according to claim 27, wherein the yeast culture is selected from the group consisting of Pichia sp., Hansenula sp., Saccharomyces sp., Schizosaccharomyces sp., Yarrovia sp and Kluyveromyces sp..

29. The method according to claim 27, wherein the yeast is cultured at a temperature of approximately 28 to 30° C.

30. The method according to claim 27, wherein the yeast is cultured on a medium containing glycerol or methanol as a carbon source.

31. The method according to claim 27, wherein expression of the modified proteinase K gene is induced with methanol.

32. The method according to claim 27, wherein the protein with the activity of Tritirachium album proteinase K is isolated from the culture fluid by hydrophobic interaction chromatography or by chromatography on a hydroxyapatite bed, and purified by dialysis against ammonium carbonate solution and freeze-dried.


SEQUENCE LISTING (22 sequences)

SEQ ID NO: 1 - protein, 384 aa, Tritirachium album
MRLSVLLSLLPSSLGAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 2 - protein, 15 aa, Tritirachium album
MRLSVLLSLLPSSLG

SEQ ID NO: 3 - protein, 90 aa, Tritirachium album
APAVEQRSEAAPLIEARGEMVANKYIVKFKEGSALSALDAAMEKISGKPDHVYKNVFSGFAATLDENMVRVLRAHPDVEYIEQDAVVTIN

SEQ ID NO: 4 - protein, 279 aa, Tritirachium album
AAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 5 - DNA, 1173 bp, Tritirachium album
  source 1..1173: mol_type = genomic DNA
gaattcgaaacgatgagactctccgtcctcctctccctcctcccctcgagtctgggcgctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggcttaaggtacc

SEQ ID NO: 6 - DNA, 41 bp, synthetic construct
  misc_feature 1..41: note = primer ProtK-G-F
  source 1..41: mol_type = other DNA
caggcgctcttctatggctccagctgttgaacaaagatctg

SEQ ID NO: 7 - DNA, 41 bp, synthetic construct
  misc_feature 1..41: note = primer ProtK-G-R
  source 1..41: mol_type = other DNA
gcagcgctcttctaccagcctggtagttgttgtaagccaac

SEQ ID NO: 8 - DNA, 1139 bp, synthetic construct
  misc_feature 1..1139: note = pro-ProtK-G DNA
  source 1..1139: mol_type = other DNA
caggcgctcttctatggctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggctggtagaagagcgctgc

SEQ ID NO: 9 - protein, 370 aa, synthetic construct
  REGION 1..370: note = pro-ProtK-G protein
APAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQAG

SEQ ID NO: 10 - DNA, 7667 bp, synthetic construct
  misc_feature 1..7667: note = pPink_HC plasmid
  source 1..7667: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacggaattccggaccggcatgccaaggcctcaggtaccggccggccatttaaatacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctggatccgcggccgccttccaaactctcatggattctcaggtaataggtattctaggaggaggccagctaggccgaatgattgttgaggccgctagcaggctcaatatcaagaccgtgattcttgatgatggtttttcacctgctaagcacattaatgctgcgcaagaccacatcgacggatcattcaaagatgaggaggctatcgccaagttagctgccaaatgtgatgttctcactgtagagattgagcatgtcaacacagatgctctaaagagagttcaagacagaactggaatcaagatatatcctttaccagagacaatcgaactaatcaaggataagtacttgcaaaaggaacatttgatcaagcacaacatttcggtgacaaagtctcagggtatagaatctaatgaaaaggcgctgcttttgtttggagaagagaatggatttccatatctgttgaagtcccggactatggcttatgatggaagaggcaattttgtagtggagtctaaagaggacatcagtaaggcattagagttcttgaaagatcgtccattgtatgccgagaagtttgctccttttgttaaagaattagcggtaatggttgtgagatcactggaaggcgaagtattctcctacccaaccgtagaaactgtgcacaaggacaatatctgtcatattgtgtatgctccggccagagttaatgacaccatccaaaagaaagctcaaatattagctgaaaacactgtgaagactttcccaggcgctggaatcttcggagttgagatgttcctattgtctgatggagaacttcttgtaaatgagattgctccaaggccccacaattctggtcactatacaatcgatgcatgtgtaacatctcagttcgaagcacatgtaagagccataactggtctgccaatgccactagatttcaccaaactatctacttccaacaccaacgctattatgctcaatgttttgggtgctgaaaaatctcacggggaattagagttttgtagaagagccttagaaacacccggtgcttctgtatatctgtacggaaagaccacccgattggctcgtaagatgggtcatatcaacataataggatcttccatgttggaagcagaacaaaagttagagtacattctagaagaatcaacccacttaccatccagtactgtatcagctgacactaaaccgttggttggagttatcatgggttcagactctgatctacctgtgatttcgaaaggttgcgatattttaaaacagtttggtgttccattcgaagttactattgtctctgctcatagaacaccacagagaatgaccagatatgcctttgaagccgctagtagaggtatcaaggctatcattgcaggtgctggtggtgctgctcatcttccaggaatggttgctgccatgactccgttgccagtcattggtgttcctgtcaagggctctacgttggatggtgtagactcgctacactcgattgtccaaatgcctagaggtgttcctgtggctacggttgctatcaacaacgccaccaatgccgctctgttggccatcaggattttaggtacaattgaccacaaatggcaaaaggaaatgtccaagtatatgaatgcaatggagaccgaagtgttggggaaggcatccaacttggaatctgaagggtatgaatcctatttgaagaatcgtctttgaatttagtattgttttttaatagatgtatatataatagtacacgtaacttatctattccattcataattttattttaaaggttcggtagaaatttgtcctccaaaaagttggttagagcctggcagttttgataggcattattatagattgggtaatatttaccctgcacctggaggaactttgcaaagagcctcatgtgcggcgcgccaggccataatggccaaacggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatggtttggccataatggcctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattg

SEQ ID NO: 11 - DNA, 8805 bp, synthetic construct
  misc_feature 1..8805: note = pPink_HC-ProtK-WT plasmid
  source 1..8805: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacggaattcgaaacgatgagactctccgtcctcctctccctcctcccctcgagtctgggcgctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggcttaaggtaccggccggccatttaaatacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctggatccgcggccgccttccaaactctcatggattctcaggtaataggtattctaggaggaggccagctaggccgaatgattgttgaggccgctagcaggctcaatatcaagaccgtgattcttgatgatggtttttcacctgctaagcacattaatgctgcgcaagaccacatcgacggatcattcaaagatgaggaggctatcgccaagttagctgccaaatgtgatgttctcactgtagagattgagcatgtcaacacagatgctctaaagagagttcaagacagaactggaatcaagatatatcctttaccagagacaatcgaactaatcaaggataagtacttgcaaaaggaacatttgatcaagcacaacatttcggtgacaaagtctcagggtatagaatctaatgaaaaggcgctgcttttgtttggagaagagaatggatttccatatctgttgaagtcccggactatggcttatgatggaagaggcaattttgtagtggagtctaaagaggacatcagtaaggcattagagttcttgaaagatcgtccattgtatgccgagaagtttgctccttttgttaaagaattagcggtaatggttgtgagatcactggaaggcgaagtattctcctacccaaccgtagaaactgtgcacaaggacaatatctgtcatattgtgtatgctccggccagagttaatgacaccatccaaaagaaagctcaaatattagctgaaaacactgtgaagactttcccaggcgctggaatcttcggagttgagatgttcctattgtctgatggagaacttcttgtaaatgagattgctccaaggccccacaattctggtcactatacaatcgatgcatgtgtaacatctcagttcgaagcacatgtaagagccataactggtctgccaatgccactagatttcaccaaactatctacttccaacaccaacgctattatgctcaatgttttgggtgctgaaaaatctcacggggaattagagttttgtagaagagccttagaaacacccggtgcttctgtatatctgtacggaaagaccacccgattggctcgtaagatgggtcatatcaacataataggatcttccatgttggaagcagaacaaaagttagagtacattctagaagaatcaacccacttaccatccagtactgtatcagctgacactaaaccgttggttggagttatcatgggttcagactctgatctacctgtgatttcgaaaggttgcgatattttaaaacagtttggtgttccattcgaagttactattgtctctgctcatagaacaccacagagaatgaccagatatgcctttgaagccgctagtagaggtatcaaggctatcattgcaggtgctggtggtgctgctcatcttccaggaatggttgctgccatgactccgttgccagtcattggtgttcctgtcaagggctctacgttggatggtgtagactcgctacactcgattgtccaaatgcctagaggtgttcctgtggctacggttgctatcaacaacgccaccaatgccgctctgttggccatcaggattttaggtacaattgaccacaaatggcaaaaggaaatgtccaagtatatgaatgcaatggagaccgaagtgttggggaaggcatccaacttggaatctgaagggtatgaatcctatttgaagaatcgtctttgaatttagtattgttttttaatagatgtatatataatagtacacgtaacttatctattccattcataattttattttaaaggttcggtagaaatttgtcctccaaaaagttggttagagcctggcagttttgataggcattattatagattgggtaatatttaccctgcacctggaggaactttgcaaagagcctcatgtgcggcgcgccaggccataatggccaaacggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatggtttggccataatggcctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattg

SEQ ID NO: 12 - protein, 460 aa, synthetic construct
  REGION 1..460: note = ProtK-G protein
MRFPSIFTAVLFAASSALAAPVNTTTEDETAQIPAEAVIGYSDLEGDFDVAVLPFSNSTNNGLLFINTTIASIAAKEEGVSLEKREAEAMAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQAG

SEQ ID NO: 13 - protein, 90 aa, synthetic construct
  REGION 1..90: note = ProtK-G SP protein
MRFPSIFTAVLFAASSALAAPVNTTTEDETAQIPAEAVIGYSDLEGDFDVAVLPFSNSTNNGLLFINTTIASIAAKEEGVSLEKREAEAM

SEQ ID NO: 14 - DNA, 1380 bp, synthetic construct
  misc_feature 1..1380: note = ProtK-G DNA
  source 1..1380: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatggctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggctggt

SEQ ID NO: 15 - DNA, 3837 bp, synthetic construct
  misc_feature 1..3837: note = pD912 plasmid
  source 1..3837: mol_type = other DNA
gctcttcaggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatgtgaagagc

SEQ ID NO: 16 - DNA, 4928 bp, synthetic construct
  misc_feature 1..4928: note = pD912-ProtK-G plasmid
  source 1..4928: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatggctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggctggttaaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 17 - protein, 459 aa, Tritirachium album
MRFPSIFTAVLFAASSALAAPVNTTTEDETAQIPAEAVIGYSDLEGDFDVAVLPFSNSTNNGLLFINTTIASIAAKEEGVSLEKREAEAMAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 18 - DNA, 59 bp, synthetic construct
  misc_feature 1..59: note = ProtK-G-out-F primer
  source 1..59: mol_type = other DNA
gcttacaacaactaccaggcttgataaaggggcggccgctcaagaggatgtcagaatgc

SEQ ID NO: 19 - DNA, 36 bp, synthetic construct
  misc_feature 1..36: note = ProtK-G-out-R primer
  source 1..36: mol_type = other DNA
gccgcccctttatcaagcctggtagttgttgtaagc

SEQ ID NO: 20 - DNA, 1380 bp, Tritirachium album
  source 1..1380: mol_type = genomic DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatggctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggcttga

SEQ ID NO: 21 - DNA, 4928 bp, synthetic construct
  misc_feature 1..4928: note = pD912-ProtK plasmid
  source 1..4928: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctatggctccagctgttgaacaaagatctgaagctgctccattgatcgaggctagaggtgaaatggttgctaacaagtacatcgttaagttcaaagagggttctgctttgtcagctttggacgctgctatggaaaagatctctggtaagccagaccacgtttacaagaacgttttctccggtttcgctgctactttggacgagaacatggttagagttttgagagctcacccagacgttgagtacattgagcaagacgctgttgttactatcaacgctgctcagactaacgctccatggggtttggctagaatttcttctacttccccaggtacttccacttactactacgacgaatctgctggtcagggttcctgtgtttacgttatcgacactggtattgaggcttcccacccagaatttgagggtagagctcagatggttaagacttactattactcctccagagatggtaacggtcacggtactcattgtgctggtactgttggttccagaacttacggtgttgctaagaaaactcagttgttcggtgttaaggttttggacgacaacggttccggtcagtactccactattattgctggtatggacttcgttgcttccgacaagaacaacagaaactgtccaaagggtgttgttgcttccttgtctttgggtggtggttactcttcttccgttaactctgctgctgctagattgcagtcctccggtgttatggttgctgttgctgctggtaacaacaacgctgacgctagaaactactctccagcttctgaaccatccgtttgtacagttggtgcttctgacagatacgacagaagatcctccttctccaactacggttccgttttggacattttcggtcctggaacttccatcttgtccacttggattggtggttccactagatccatttccggtacttctatggctactccacacgttgctggattggctgcttacttgatgactttgggtaagactactgctgcttctgcttgtagatatatcgctgacacagctaacaagggtgacttgtccaacatcccattcggtactgttaacttgttggcttacaacaactaccaggcttgataaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 22 - protein, 280 aa, Tritirachium album
AAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQAG